(19) United States
(12) Patent Application Publication
(10) Pub. No.: US 2022/0096343 A1
(43) Pub. Date: Mar. 31, 2022

(54) COSMETIC PRODUCT USING PHOTOCHROMIC POWDER AND METHOD OF MANUFACTURING THE SAME

(71) Applicant: BBM Korea Co., Ltd., Incheon (KR)

(72) Inventor: Yun-ha KIM, Incheon (KR)

(21) Appl. No.: 16/623,742

(22) PCT Filed: Jul. 1, 2019
(86) PCT No.: PCT/KR2019/007951

§ 371 Date: Nov. 12, 2021

(30) Foreign Application Priority Data
Nov. 16, 2018 (KR) 10-2018-0141379
Mar. 5, 2019 (KR) 10-2019-0025392

(51) Int. Cl. A61K 8/31; A61K 8/81; A61K 8/49; A61K 8/36; A61Q 1/06; A61Q 5/00; A61Q 3/02

(52) U.S. Cl. CPC A61K 8/31; A61K 8/8117; A61K 8/494; A61K 8/361; A61Q 1/06; A61K 2800/805; A61Q 3/02; A61K 2800/438; A61K 2800/591; A61K 2800/624; A61K 2800/412; A61Q 5/00

(57) ABSTRACT

The present invention relates to a cosmetic product using photochromic powder and a method of manufacturing the same. More particularly, the present invention relates to a cosmetic product using photochromic powder and a method of manufacturing the same, in which various formulations of cosmetic products used for the skin, lips, nails, and hair are manufactured using a photochromic powder pigment manufactured using ingredients that are harmless to the human body so that the color thereof is changed when the cosmetic products are exposed to ultraviolet light, thereby making it possible to satisfy user demand both for fun and for aesthetic improvement and to help a user create a unique style.

### TECHNICAL FIELD

The present invention relates to a cosmetic product using photochromic powder and a method of manufacturing the same. More particularly, the present invention relates to a cosmetic product using photochromic powder and a method of manufacturing the same, in which various formulations of cosmetic products used for the skin, lips, nails, and hair are manufactured using a photochromic powder pigment manufactured using ingredients that are harmless to the human body so that the color thereof is changed when the cosmetic products are exposed to ultraviolet light, thereby making it possible to satisfy user demand both for fun and for aesthetic improvement and to help a user create a unique style.

### BACKGROUND ART

A photochromic raw material is a compound material that changes the molecular structure thereof upon absorbing light energy having a specific wavelength to, thus changing the outwardly apparent color thereof. In this regard, when the energy is not applied to the above-described material, the molecular structure thereof reversibly returns to the original molecular structure.

Specifically, in the case of the photochromic raw material, a ring structure in the molecule is converted into a conjugated structure when the amount of ultraviolet light that is absorbed is increased, so that the wavelength of the reflected light is changed to thus change the color thereof. On the other hand, when the amount of ultraviolet light that is absorbed is reduced, the molecular structure is converted back into the ring structure, thus returning the color to the original color, whereby reversibility is exhibited.

Such photochromic raw material may be generally applied both to paint ink and to plastics. Recently, photochromic raw material has come to be used in various fields such as those of toys, including dolls, sunglasses, crayons, hair clips, belts, nail polish, greeting cards, stickers, and T-shirts.

However, conventional photochromic raw materials used as described above are mainly diarylethene-based photochromic raw materials, do not dissolve without an organic solvent, contain a lot of components that are harmful to the human body, and do not react with natural plant ingredients. Accordingly, conventional photochromic raw materials are not used as raw materials for cosmetic products.

Therefore, there is a need to develop a novel photochromic raw material that includes a combination of materials that are not harmful to the human body and is excellent in reactivity to ultraviolet light, thus being useful as a raw material of a cosmetic product.

### DISCLOSURE

### Technical Problem

Accordingly, the present invention has been made keeping in mind the above problems occurring in the prior art, and an object of the present invention is to provide a cosmetic product using photochromic powder and a method of manufacturing the same, in which various formulations of cosmetic products used for the skin, lips, nails, and hair are manufactured using a photochromic powder pigment manufactured using ingredients that are harmless to the human body so that the color thereof is changed when the cosmetic products are exposed to ultraviolet light, thereby making it possible to satisfy user demand both for fun and for aesthetic improvement and to help a user create a unique style.

### Technical Solution

In order to accomplish the above objects, a cosmetic product using a photochromic powder according to the present invention includes

80 to 97 wt % of a base raw material of the cosmetic product and 3 to 20 wt % of the photochromic powder.

The photochromic powder is a micro-encapsulated photochromic powder in which a raw material of the photochromic powder is surrounded by a polyoxymethylenemelamine-styrene copolymer, the raw material of the photochromic powder includes 47 to 49 parts by weight of xylyl phenylethane, 13 to 23 parts by weight of pentaerythrityl tetra-di-t-butyl hydroxyhydrocinnamate, and 2 to 4 parts by weight of a reversible photochromic powder, and the weight ratio of styrene to polyoxymethylenemelamine in the polyoxymethylenemelamine-styrene copolymer is in the range of 1:6 to 6:1.

Further, the reversible photochromic powder is any one among trimethyl trifluoromethyl indolinopiperidinyl spironaphtoxazine, indolinotrimethyl spiroindolinenaphtoxazine, trimethyl indolinopiperidinyl spironaphtoxazine, and diphenyl naphthopyran.

In addition, the base raw material of the cosmetic product is any one base raw material among lip balm, lipstick, lip gloss, tint, hairspray, and nail polish.

When the base raw material of the cosmetic product is a base raw material of the lip balm, the lip balm includes 80 wt % of the base raw material of the lip balm and 20 wt % of the photochromic powder.

Further, when the base raw material of the cosmetic product is a base raw material of the lipstick, the lipstick includes 85 wt % of the base raw material of the lipstick and 15 wt % of the photochromic powder.

In addition, when the base raw material of the cosmetic product is a base raw material of the lip gloss or the tint, the lip gloss or the tint includes a combination of 93 wt % of the base raw material of the lip gloss or the tint and 7 wt % of the photochromic powder.

Further, when the base raw material of the cosmetic product is a base raw material of the hairspray or the nail polish, the hairspray or the nail polish includes a combination of 97 wt % of the base raw material of the hairspray or the nail polish and 3 wt % of the photochromic powder.

Meanwhile, a method of manufacturing a cosmetic product using a photochromic powder according to the present invention includes

a base-raw-material addition step of adding a base raw material of the cosmetic product, which is synthesized by combining raw materials used to manufacture the cosmetic product, to a container, a photochromic-powder addition step of adding the manufactured photochromic powder to a container, a raw-material-mixing step of mixing the base raw material of the cosmetic product and the photochromic powder that are added using a homomixer, and a finishing-work step of quantitatively adding the mixed raw materials to a container for cosmetic products, thereby completing manufacture thereof.

The base raw material of the cosmetic product added in the base-raw-material addition step is any one base raw material among lip balm, lipstick, lip gloss, tint, hairspray, and nail polish.

Further, the method further includes, when the base raw material of the cosmetic product added in the base-raw-material addition step is the base raw material of the lip balm or the lipstick, a cooling step of adding raw materials mixed after a raw-material-mixing step to a quick cooler to cool the raw materials.

In addition, the photochromic powder added in the photochromic-powder addition step is manufactured using a process that includes (a) a material addition step of adding materials including xylyl phenylethane, pentaerythrityl tetra-di-t-butyl hydroxyhydrocinnamate, and a reversible photochromic powder, followed by mixing, (b) a material-mixing step of adding acetone to the materials added in the material addition step and performing mixing so that the weight ratio of the materials and the acetone added is in the range of 2:1 to 1:2, (c) a microcapsule-material addition step of adding a microcapsule material surrounding an external part of the raw material of the photochromic powder mixed in the material-mixing step, (d) a curing-agent-addition step of adding a curing agent for curing the microcapsule material, (e) an agitation step of performing agitation at a high temperature of 100 to 130° C. in order to age the microcapsules to which the curing agent is added and an aging step of performing aging in order to stabilize the physical properties thereof, (f) a sterilization and disinfection step of cleaning the microcapsules, which are aged after the aging step, using ethanol, (g) a photochromic-powder-manufacturing step of drying the cleaned microcapsules and then pulverizing the dried microcapsules into fine particles using an agitator, and (h) a final inspection step of finally inspecting the cleanliness and glossiness of the manufactured photochromic powder.

The reversible photochromic powder is any one among trimethyl trifluoromethyl indolinopiperidinyl spironaphtoxazine, indolinotrimethyl spiroindolinenaphtoxazine, trimethyl indolinopiperidinyl spironaphtoxazine, and diphenyl naphthopyran.

Further, a microcapsule material added in step (c) is a polyoxymethylenemelamine-styrene copolymer, and the weight ratio of styrene to polyoxymethylenemelamine in the polyoxymethylenemelamine-styrene copolymer is in a range of 1:6 to 6:1.

In addition, a photochromic powder manufactured in step (g) is micro-encapsulated fine particles having a size of 1 to 10 micrometers.

Further, in step (a), 47 to 49 parts by weight of xylyl phenylethane, 13 to 23 parts by weight of pentaerythrityl tetra-di-t-butyl hydroxyhydrocinnamate, and 2 to 4 parts by weight of the reversible photochromic powder are added.

Further, the curing agent added in step (d) is stearic acid.

### Advantageous Effects

According to the present invention, it is possible to manufacture various cosmetic products capable of being used for the skin, lips, hair, and nails using photochromic powder which includes a combination of ingredients harmless to the human body and which has excellent reversibility, so that the photochromic powder reacts with ultraviolet light to thus change in color and the color thereof returns to the original color when the ultraviolet light is not radiated thereon.

Further, according to the present invention, various formulations of cosmetic products are manufactured using photochromic powder so that the color thereof is changed when the cosmetic products are exposed to ultraviolet light, thereby making it possible to satisfy user demand both for fun and for aesthetic improvement and to help a user create a unique style.

### BRIEF DESCRIPTION OF DRAWINGS

FIG. 1 is a flowchart showing a process of manufacturing a cosmetic product using photochromic powder according to the present invention;

FIG. 2 is a view showing the photochromic powder used in the present invention;

FIG. 3 is a flowchart showing a process of manufacturing the photochromic powder used in the present invention;

FIGS. 4A and 4B are views showing a change in the color of lip balm, among cosmetic products using the photochromic powder according to the present invention;

FIGS. 5A and 5B are views showing a change in the color of lipstick, among cosmetic products using the photochromic powder according to the present invention;

FIGS. 6A and 6B are views showing a change in the color of lip gloss and tint, among cosmetic products using the photochromic powder according to the present invention;

FIGS. 7A and 7B are views showing a change in the color of hairspray, among cosmetic products using the photochromic powder according to the present invention; and

FIG. 8 is a view showing a change in the color of nail polish, among cosmetic products using the photochromic powder according to the present invention.

### BEST MODE

Hereinafter, preferred embodiments of photochromic powder and a method of manufacturing the same according to the present invention will be described in detail with reference to the accompanying drawings.

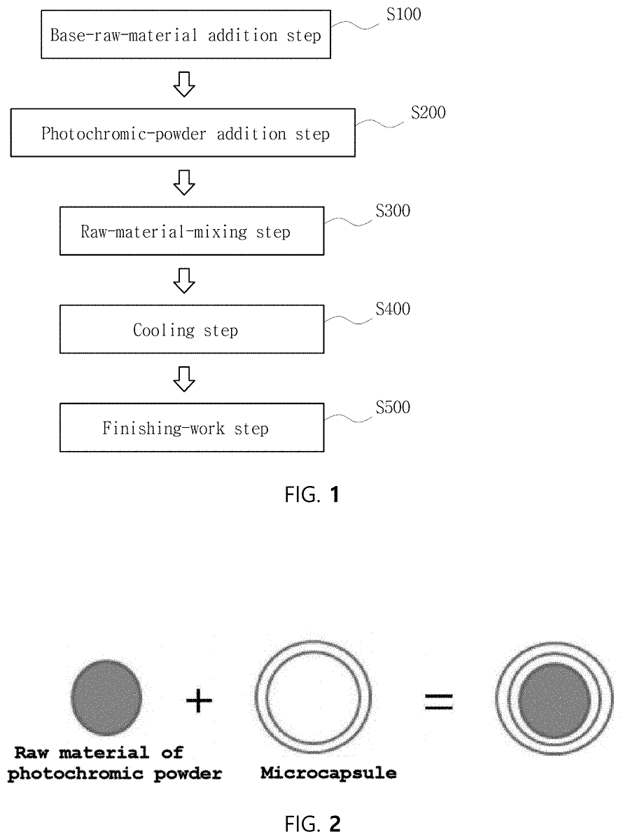

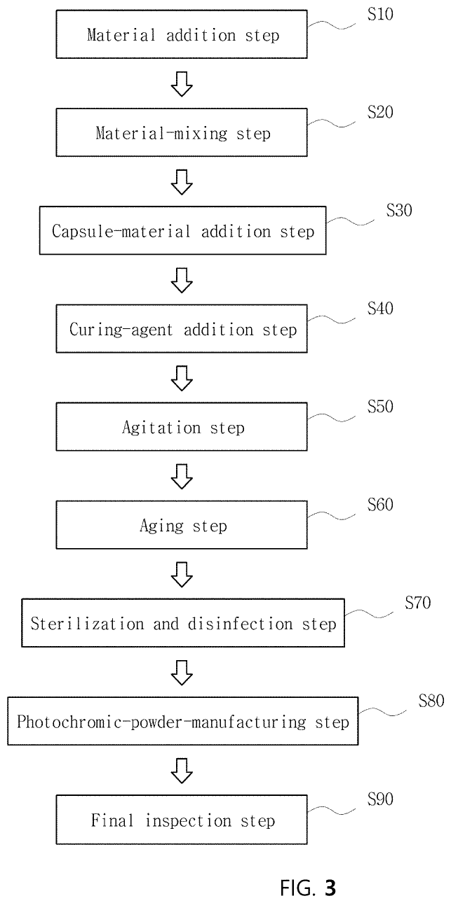

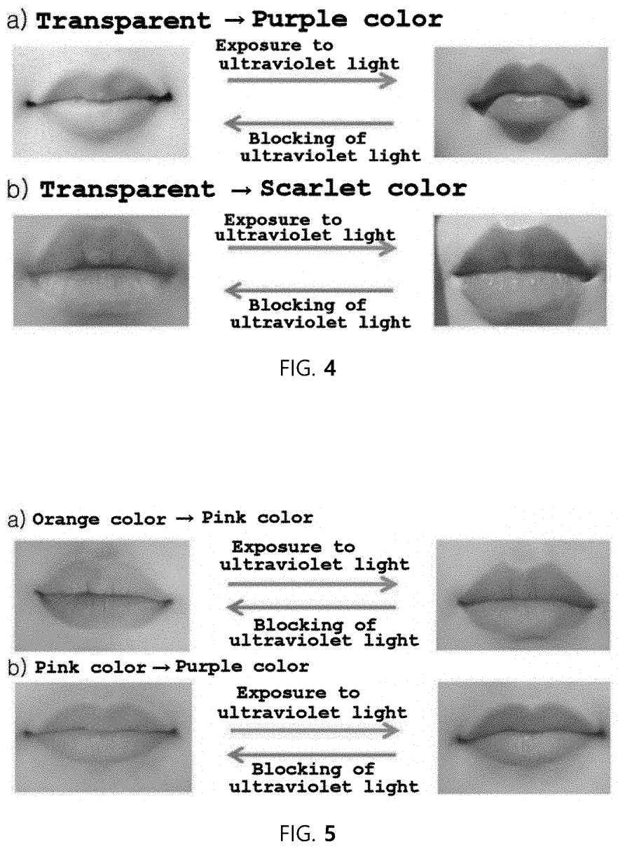

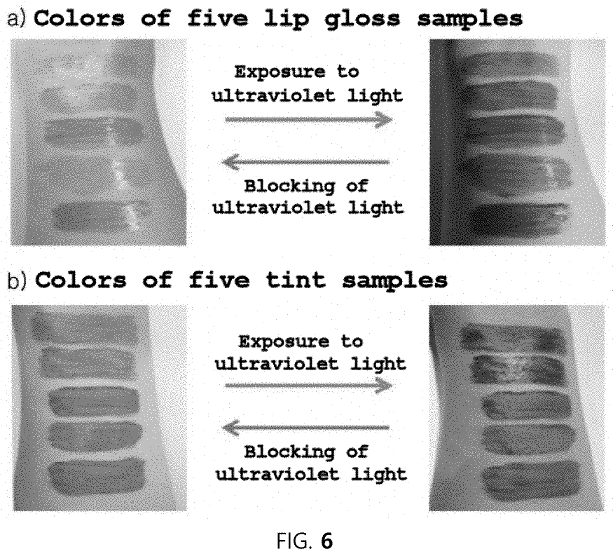

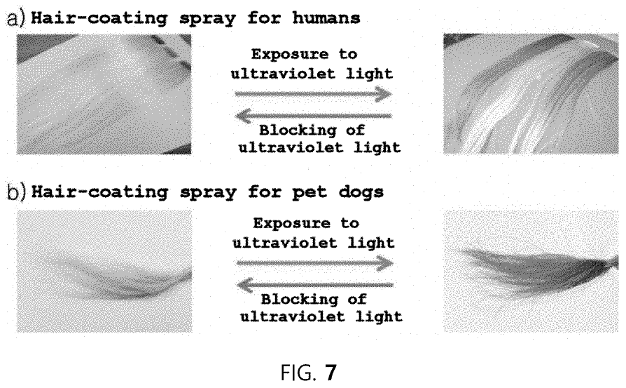

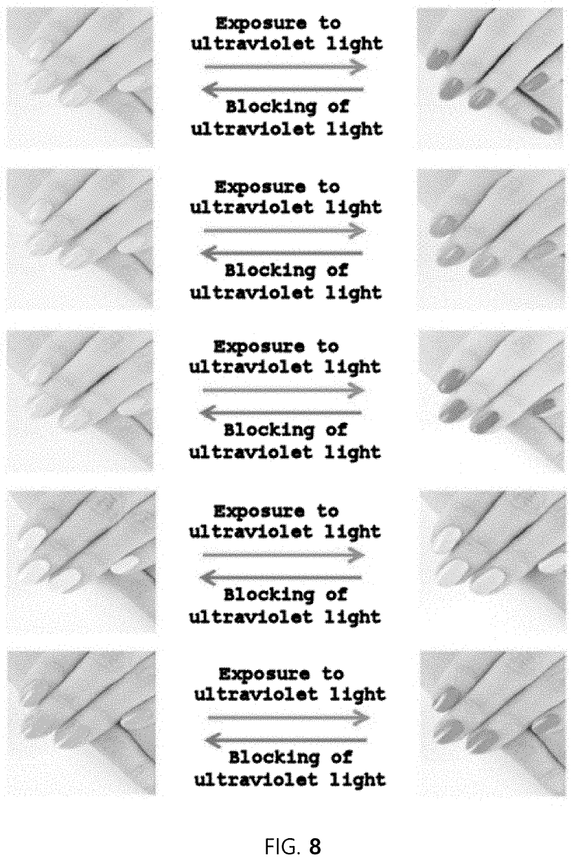

FIG. 1 is a flowchart showing a process of manufacturing a cosmetic product using photochromic powder according to the present invention, FIG. 2 is a view showing the photochromic powder used in the present invention, FIG. 3 is a flowchart showing a process of manufacturing the photochromic powder used in the present invention, FIGS. 4A and 4B are views showing a change in the color of lip balm, among cosmetic products using the photochromic powder according to the present invention, FIGS. 5A and 5B are views showing a change in the color of lipstick, among cosmetic products using the photochromic powder according to the present invention, FIGS. 6A and 6B are views showing a change in the color of lip gloss and tint, among cosmetic products using the photochromic powder according to the present invention, FIGS. 7A and 7B are views showing a change in the color of hairspray, among cosmetic products using the photochromic powder according to the present invention, and FIG. 8 is a view showing a change in the color of nail polish, among cosmetic products using the photochromic powder according to the present invention.

The present invention relates to a cosmetic product using photochromic powder and a method of manufacturing the same, in which various formulations of cosmetic products used for the skin, lips, nails, and hair are manufactured using a photochromic powder pigment manufactured using ingredients that are harmless to the human body so that the color thereof is changed when the cosmetic products are exposed to ultraviolet light, thereby making it possible to satisfy user demand both for fun and for aesthetic improvement and to help a user create a unique style. First, the cosmetic product using the photochromic powder according to the present invention includes a combination of 80 to 97 wt % of a base raw material of the cosmetic product and 3 to 20 wt % of the photochromic powder.

The base raw material of the cosmetic product includes a mixture of ingredients used to manufacture cosmetic products used for the skin, lips, nails, and hair of the human body. For example, the base raw material of the cosmetic product may be used as the base raw materials of various formulations of cosmetic products such as lip balm, lipstick, lip gloss, tint, hairspray, and nail polish, and as the base raw materials of pet products such as spray for pet dogs.

Next, the photochromic powder is obtained by shaping a photochromic raw material, which has reversibility whereby the color thereof is changed when the photochromic powder is exposed to ultraviolet light and the new color returns to the original color when the ultraviolet light is not radiated thereon, into powder using ingredients that are harmless to the human body and performing micro-encapsulation. Accordingly, the photochromic powder may have excellent stability and may be readily applied to aqueous and natural ingredients, thus being freely usable for any cosmetic product formulation.

Further, when cosmetic products are manufactured using the photochromic powder as in the present invention, there is an additional merit in that it is not necessary to use a solvent, which is a harmful ingredient.

In more detail, the raw material of the photochromic powder used in the present invention includes xylyl phenylethane, pentaerythrityl tetra-di-t-butyl hydroxyhydrocinnamate, and a reversible photochromic powder. The raw material of the photochromic powder may further include cosmetic materials such as vitamin, flavoring agents, and natural ingredients.

Further, the base cosmetic material used in the present invention may include waxes, oils, skin moisturizers, flavoring agents, acids, viscosity thickeners, nutritional ingredients, volatile solubilizers, antioxidants, or preservatives. The wax is preferably solid-phase wax, and may be selected from the group consisting of microcrystalline wax, ozokerite, beeswax, candelilla wax, carnauba wax, ceresin, paraffin, montan wax, Japan wax, cocoa butter, and synthetic wax, but is not limited thereto.

Examples of the oil ingredient may include mineral oil such as liquid paraffin, animal oil such as squalene and hydrosqualene, vegetable oil such as palm oil, avocado oil, jojoba oil, sesame oil, olive oil, and castor oil, and synthetic oil such as butyl myristate, isopropyl myristate, cetyl myristate, isopropyl palmitate, butyl stearate, polybutene, polyisobutene, hydrogenated polyisobutene, various polyols, glycerin tri-2-ethylhexylate, triglyceride-based oil, myristyl lactate, octyl dodecanol, and diisostearyl maleate, but are not limited thereto.

The skin moisturizer contains aqueous and moisturizing ingredients, thus ensuring dual moisturizing ability of supplying moisture and preventing moisture from evaporating. It is preferable that the skin moisturizer be one or more selected from the group consisting of betaine, panthenol, trehalose, dipropylene glycol, propylene glycol, and glycerin.

The acid additionally provides nutrients and moisturizing ingredients. It is preferable that the acid be one or more selected from the group consisting of glycolic acid, acetic acid, and salicylic acid.

The viscosity thickener is an ingredient that increases the viscosity of the final product and plays an important role in influencing the stability, usability, and sensation of use of the product. Since the viscosity thickener is contained therein, the viscosity of the product is capable of being adjusted, and it is preferable that the viscosity thickener be one or more selected from the group consisting of potassium alginate and cellulose gum.

First, the xylyl phenylethane is a kind of oil wax, and serves to increase the color visibility of the raw material of the photochromic powder.

That is, the xylyl phenylethane acts as both the oil and the wax. The xylyl phenylethane is used as an emulsifying agent, and serves to improve water and pollution resistances of the raw material of the photochromic powder and also to increase the visibility thereof.

The xylyl phenylethane is used in a content of 47 to 49 parts by weight based on the entirety of the raw material of the photochromic powder. When the content is less than 47 parts by weight, there is the concern that the above-described functions of the xylyl phenylethane will not be properly exhibited. When the content is more than 49 parts by weight, the content of other components is reduced, so that the desired effect is less likely to be realized.

Next, the pentaerythrityl tetra-di-t-butyl hydroxyhydrocinnamate is used as an antioxidant, and serves to prevent deterioration of the raw material of the photochromic powder, thus improving durability.

The pentaerythrityl tetra-di-t-butyl hydroxyhydrocinnamate may be used in a content of 13 to 23 parts by weight based on the entirety of the raw material of the photochromic powder. When the content is less than 13 parts by weight, there is the concern that the above-described durability improvement effect will not be properly exhibited. When the content is more than 23 parts by weight, the content of other components is reduced, so that the desired effect is less likely to be realized.

Next, the reversible photochromic powder is changed in color when the photochromic powder is exposed to ultraviolet light and enables the new color to return to the original color when ultraviolet light is not radiated thereon. The photochromic powder is transparent and colorless when the ultraviolet light is not radiated thereon, and is changed to be colored when the photochromic powder is exposed to ultraviolet light.

That is, when the reversible photochromic powder is affected by ultraviolet (UV) light, the photochromic molecules lose their twisted shape and come untwisted, so that the powder is converted to the powder present in an equilibrium state, showing a bright color.

The untwisted state of the photochromic molecules enables visible rays to be very effectively absorbed, and this new color returns to the color corresponding to the inactive or resting state of the molecules when the source of radiation is removed.

The reaction rate of the reversible photochromic powder depends on the ambient temperature and the chemical structure thereof. In the case of the reaction wavelength, color formation occurs in both the normal UV-A and UV-B wavelength ranges. The color is exhibited within about 1 second, and when UV-A and UV-B are not radiated, the color returns to the original color within about 20 seconds.

The reversible photochromic powder may be used in a content of 2 to 4 parts by weight based on the entirety of the raw material of the photochromic powder. When the content is less than 2 parts by weight, there is the concern that the change in color caused by exposure to ultraviolet light will not be vividly exhibited. When the content is more than 4 parts by weight, since the content of the reversible photochromic powder becomes higher than necessary, there is the concern that manufacturing costs will be increased.

Meanwhile, examples of the reversible photochromic powder may include any one among trimethyl trifluoromethyl indolinopiperidinyl spironaphtoxazine, indolinotrimethyl spiroindolinenaphtoxazine, trimethyl indolinopiperidinyl spironaphtoxazine, and diphenyl naphthopyran, or a combination of two or more thereof. First, the trimethyl trifluoromethyl indolinopiperidinyl spironaphtoxazine corresponds to CAS No. 172208-34-3, and is in the form of powder having a yellow color and a melting point of about 140° C. The color thereof is changed to a red color by ultraviolet light.

Next, the indolinotrimethyl spiroindolinenaphtoxazine corresponds to CAS No. 114747-44-3, and is in the form of powder having a yellow color and a melting point of about 160° C. The color thereof is changed to a blue color by ultraviolet light. Next, the trimethyl indolinopiperidinyl spironaphtoxazine corresponds to CAS No. 114747-45-4, and is in the form of powder having a yellow color and a melting point of about 140° C. The color thereof is changed to a purple color by ultraviolet light.

Next, the diphenyl naphthopyran corresponds to CAS No. 4222-20-2, and is in the form of powder having a white color and a melting point of about 140° C. The color thereof is changed to an orange or yellow color by ultraviolet light.

All four types of reversible photochromic powder are colored in UV-A and UV-B wavelength ranges, that is, in the wavelength of about 285 to 382 nm as described above. The color is exhibited within about 1 second, and when ultraviolet light is not radiated, the color returns to the original color within about 20 to 60 seconds.

Further, all four types of the reversible photochromic powder are harmless to the human body and are easily dissolved in water, so the reversible photochromic powders may be easily used in the synthesis of any cosmetic product formulation. Since the reversible photochromic powders are capable of being mixed with general colored raw materials, both a change from colorlessness to a color and a color change from one color to another color are possible. Accordingly, a variety of changes in color may be realized.

Further, the photochromic powder may further include a plasticizer and a fatty acid. Examples of the fatty acid may include palmitic acid, oleic acid, linoleic acid, and linolenic acid, and examples of the plasticizer may include 1,3-butanediol.

The plasticizer is used to swell the polyoxymethylenemelamine-styrene copolymer, which is a shell material, and is used in a content of 0.1 to 1 parts by weight, which is a range within which physical properties are not impaired. The fatty acid is used to improve the physical properties of the polyoxymethylenemelamine-styrene copolymer, and it is preferable to use the fatty acid in a content in the range of 0.1 to 1 part by weight.

The polyoxymethylenemelamine-styrene copolymer is used as the microcapsule material, which is the material surrounding the photochromic powder. The polyoxymethylenemelamine-styrene copolymer is a film-forming agent, that is, acts as a shell surrounding the photochromic raw material. In the polyoxymethylenemelamine-styrene copolymer, the weight ratio of styrene to polyoxymethylenemelamine is preferably in the range of 1:6 to 6:1. More preferably, the weight ratio is in the range of 1:6 to 1:2.

When the weight ratio in the polyoxymethylenemelamine-styrene copolymer is outside the above-described range, the mixing and synthesizing effects are reduced. When the weight ratio in the polyoxymethylenemelamine-styrene copolymer is in the above-described range, the change in color caused by exposure to ultraviolet light may appear more strongly. When the weight ratio in the polyoxymethylenemelamine-styrene copolymer is outside the above-described range, there is the concern that the colors will not be clearly distinguished due to the very easy color change. It is more preferable to use the polyoxymethylenemelamine in a content of 19 to 27 parts by weight and the styrene in a content of 5 to 7 parts by weight.

Meanwhile, as described above, the cosmetic product using the photochromic powder according to the present invention includes a combination of 80 to 97 wt % of a base raw material of the cosmetic product and 3 to 20 wt % of the photochromic powder. The content of the base raw material of the cosmetic product and the photochromic powder may depend on the type of cosmetic product.

In more detail, when the base raw material of the cosmetic product is a base raw material of lip balm, 80 wt % of the base raw material of the lip balm and 20 wt % of the photochromic powder are mixed to manufacture the lip balm. Since lip balm is a solid, a relatively large amount of the photochromic powder is used compared to other cosmetic products that are in a liquid form.

Further, when the base raw material of the cosmetic product is a base raw material of lipstick, 85 wt % of the base raw material of the lipstick and 15 wt % of the photochromic powder are mixed to manufacture the lipstick. Since the lipstick is also a solid, a relatively large amount of the photochromic powder is used compared to other cosmetic products that are in a liquid form. However, since lipstick generally has its own intrinsic color, a relatively small amount of the photochromic powder is used compared to lip balm, which is transparent.

In addition, when the base raw material of the cosmetic product is a base raw material of the lip gloss or the tint, 93 wt % of the base raw material of the lip gloss or the tint and 7 wt % of the photochromic powder are mixed to manufacture the lip gloss or the tint. Since the base raw materials of the lip gloss and the tint are almost the same as each other, the ratios of the photochromic powders used in mixing are the same as each other. Since the lip gloss and the tint are liquids, a relatively small amount of photochromic powder is used compared to the above-described lip balm and lipstick.

Next, when the base raw material of the cosmetic product is a base raw material of hairspray or spray for pet dogs, 97 wt % of the base raw material of the hairspray or spray for pet dogs and 3 wt % of the photochromic powder are mixed to manufacture the hairspray or spray for pet dogs. Since the spray products are mostly in the form of a mixture of liquid and gas, a relatively small amount of photochromic powder is used compared to other cosmetic products.

In addition, when the base raw material of the cosmetic product is a base raw material of nail polish, 97 wt % of the base raw material of the nail polish and 3 wt % of the photochromic powder are mixed to manufacture the nail polish. Nail polish is a liquid but has its own intrinsic color, like lipstick. Accordingly, a relatively small amount of the photochromic powder may be used compared to other cosmetic products that are in a liquid form.

Meanwhile, as shown in FIG. 1, a method of manufacturing a cosmetic product using a photochromic powder according to the present invention broadly includes a base-raw-material addition step at step S100, a photochromic-powder addition step at step S200, a raw-material-mixing step at step S300, and a finishing-work step at step S500. First, the base-raw-material addition step at step S100 relates to a step of adding the base raw material of the cosmetic product, which is synthesized by combining raw materials used to manufacture the cosmetic product, to a container.

That is, the base raw material of the cosmetic product is manufactured by synthesis using a combination of the raw materials used as the materials of the cosmetic product to be manufactured, and the manufactured base raw material of the cosmetic product is added to the container.

As described above, any one base raw material among lip balm, lipstick, lip gloss, tint, hairspray, and nail polish may be used as the base raw material of the cosmetic product.

Next, the photochromic-powder addition step at step S200 relates to a step of manufacturing the photochromic powder that is changed in color depending on exposure to ultraviolet light and then adding the photochromic powder to a container. As shown in FIG. 3, the photochromic powder is manufactured using a process which includes a material addition step at step S10, a material-mixing step at step S20, a capsule-material addition step at step S30, a curing-agent-addition step at step S40, an agitation step at step S50, an aging step at step S60, a sterilization and disinfection step at step S70, a photochromic-powder-manufacturing step at step S80, and a final inspection step at step S90.

In more detail, the material addition step at step S10 relates to a step of adding materials, which are used to manufacture the raw material of the photochromic powder, that is, oil wax, an antioxidant, and a reversible photochromic powder, to a container or an agitator, followed by mixing. In other words, materials including xylyl phenylethane, pentaerythrityl tetra-di-t-butyl hydroxyhydrocinnamate, and the reversible photochromic powder are added, followed by mixing, and this step is as described above, so a detailed description thereof will be omitted.

Next, the material-mixing step at step S20 relates to a step of adding a solvent to the added materials and then mixing the materials. Acetone is used as the solvent.

The acetone serves to smoothly dissolve the added materials to perform mixing. The acetone is added so that the weight thereof is the same as the total weight of the materials added in the material addition step at step S10, that is, the weight ratio of the materials and the acetone added is 2:1 to 1:2, followed by mixing, thus manufacturing the raw material of the photochromic powder.

Next, the capsule-material addition step at step S30 relates to a step of adding a microcapsule material surrounding the external part of the raw material of the photochromic powder mixed in the material-mixing step at step S20. The microcapsules are ultrafine particles having a size of 1 to 10 μm, and surround the external part of the photochromic raw material as shown in FIG. 1, thereby protecting the photochromic raw material from materials that are unstable due to the external environment, heat, or pH and maintaining the function of the photochromic raw material.

Further, the microcapsule material is manufactured using the polyoxymethylenemelamine-styrene copolymer.

Next, the curing-agent-addition step at step S40 relates to a step of adding a curing agent so that the added capsule material is rapidly hardened. An aliphatic amine solvent or fatty acid may be used as the curing agent. More preferably, stearic acid may be used.

Next, the agitation step at step S50 relates to a step of agitating the mixture material including the photochromic raw material and the capsule material at a high temperature of about 100 to 130° C. The aging period of the material may be shortened due to the agitation process. The subsequent aging step at step S60 relates to a step of allowing the materials, mixed using the agitation at room temperature, to stand for about 24 hours to perform aging. The mixed materials may be chemically reacted through the aging process, thereby stabilizing the physical properties thereof.

Next, the sterilization and disinfection step at step S70 relates to a step of sterilizing and disinfecting the photochromic raw material micro-encapsulated after the aging is completed. The aged photochromic raw material is cleaned using ethanol at a temperature of about 70° C. for about 1 hour, thereby achieving sterilization and disinfection.

Next, the photochromic-powder-manufacturing step at step S80 relates to a step of pulverizing the photochromic raw material, which is subjected to sterilization and disinfection at step S70 by cleaning using ethanol, into fine particles using an agitator. After the moisture contained in the photochromic raw material is completely removed through a drying process for about 30 minutes, the photochromic raw material is pulverized into fine particles using the agitator, thus manufacturing photochromic powder.

Finally, the final inspection step at step S90 relates to a step of finally inspecting the cleanliness and glossiness of the manufactured photochromic powder. When the inspection result is poor, the sterilization and disinfection step and the drying process are again performed.

The photochromic powder manufactured through the above-described procedure is added to the container to which the base raw material of the cosmetic product is added. Since the weight ratio of the base raw material of the cosmetic product added in the base-raw-material addition step at step S100 and the photochromic powder added in the photochromic-powder addition step at step S200 is as described above, a detailed description thereof will be omitted.

Next, the raw-material-mixing step at step S300 relates to a step of mixing the base raw material of the cosmetic product and the photochromic powder added to the container to manufacture the cosmetic product including the photochromic powder. A homomixer is used to mix the base raw material of the cosmetic product and the photochromic powder.

In more detail, since the photochromic powder is easily dissolved in water, the photochromic powder may be mixed with the base raw material of the cosmetic product without using a solvent that is harmful to the human body. To this end, in the raw-material-mixing step at step S300, preferably, a homomixer is set at 3,000 rpm or more to perform mixing for 30 minutes or more, so that the base raw material of the cosmetic product and the photochromic powder are uniformly mixed.

Further, as described above, in the case of the raw material of the photochromic powder, a change from colorlessness to a colored state is achieved using ultraviolet light. Accordingly, in order to manufacture a cosmetic product that is changed from one color to another color, a coloring pigment may be further added, followed by mixing during the raw-material-mixing step at step S300.

Meanwhile, when the base raw material of the cosmetic product added in the base-raw-material addition step at step S100 is a base raw material of the lip balm or lipstick, that is, when the cosmetic product to be manufactured is a solid, like the lip balm or lipstick, a cooling step at step S400 may be further performed after the raw-material-mixing step at step S300. The cooling step at step S400 relates to a step of adding the liquid raw materials mixed using a homomixer in the raw-material-mixing step at step S300 to a quick cooler, so that the raw materials are cooled for about 20 minutes, thereby solidifying the mixed raw materials.

Next, the finishing-work step at step S500 relates to a step of quantitatively adding the raw materials that are mixed in the raw-material-mixing step at step S300 or the raw materials that are cooled to be solidified in the cooling step at step S400 to a container for cosmetic products, thereby completing the manufacture thereof.

### MODE FOR INVENTION

### Preparation Example 1 of Photochromic Powder

3.4 g of a polyoxymethylenemelamine-styrene copolymer as a polymer was dissolved with agitation in 10 ml of acetone as an organic solution at a temperature of 25° C., thus manufacturing a solution A. Subsequently, 4.7 g of xylyl phenylethane, 1.3 g of pentaerythrityl tetra-di-t-butyl hydroxyhydrocinnamate, and 0.2 g of trimethyl trifluoromethyl indolinopiperidinyl spironaphtoxazine, which is a reversible photochromic powder, were dispersed in 10 ml of acetone at a temperature of 25° C. for 15 minutes to manufacture a solution B. Subsequently, the solution A was added to the dispersion solution B to perform homogeneous dispersion for 30 minutes, and 1 g of stearic acid was added thereto to manufacture microcapsules. Subsequently, the manufactured microcapsules were cleaned with ethanol, and the cleaned microcapsules were dried and then pulverized using an agitator, thus manufacturing photochromic powder.

### Preparation Example 2 of Photochromic Powder

The photochromic powder was manufactured using the same method as in Preparation Example 1 of photochromic powder, except that 0.2 g of trimethyl indolinopiperidinyl spironaphtoxazine, which is the reversible photochromic powder, was used.

### Comparative Example 1 of Photochromic Powder

A photochromic powder c was manufactured using the same method as in Preparation Example 1 of the photochromic powder, except that a solution A was added.

### Comparative Example 2 of Photochromic Powder

A photochromic powder d was manufactured using the same method as in Preparation Example 2 of the photochromic powder, except that a solution A was added.

Meanwhile, FIGS. 4 to 8 show the results of a test on cosmetic products using the photochromic powders manufactured according to the present invention. FIGS. 4A and 4B show the results of a test on the lip balms respectively manufactured using trimethyl indolinopiperidinyl spironaphtoxazine (Preparation Example 2 of photochromic powder) and trimethyl trifluoromethyl indolinopiperidinyl spironaphtoxazine (Preparation Example 1 of photochromic powder) as the reversible photochromic powder.

In the case of the lip balm including the trimethyl indolinopiperidinyl spironaphtoxazine of which a color is changed to a purple color, it can be confirmed that the colorless lip balm changes to purple lip balm when the lip balm is exposed to ultraviolet light. In the case of lip balm including trimethyl trifluoromethyl indolinopiperidinyl spironaphtoxazine, the color of which changes to a red color, it can be confirmed that the colorless lip balm changes into scarlet lip balm when the lip balm is exposed to ultraviolet light. Even when the lip balm is used over a long period of time, consistent performance is maintained, which shows that the micro-encapsulated photochromic powder is little affected by the external environment or heat.

However, in the cases of the lip balm including the photochromic powder e manufactured in Comparative Example 1 and the lip balm including the photochromic powder f manufactured in Comparative Example 2, the color changed to purple and scarlet colors in the respective cases when the lip balms were exposed to ultraviolet light, but it was confirmed that the photochromic performance greatly deteriorated after 1 hour. In this regard, it could be seen that when the photochromic powder that was not micro-encapsulated was exposed to the external environment, the performance of the photochromic powder deteriorated and thus the photochromic characteristic was lost.

Similarly, FIGS. 5 to 8 show the results of tests on the effects of the lipstick, lip gloss, tint, hairspray, spray for pet dogs, and nail polish manufactured using the photochromic powders according to the present invention. It can be confirmed that all of them exhibit a vivid color change when exposed to ultraviolet light, and that both a change from colorlessness to a colored state and a color change from one color to another color are possible through mixing with general colored raw materials, thereby exhibiting various color changes.

Therefore, according to a cosmetic product using photochromic powder and a method of manufacturing the same according to the present invention, the photochromic powder includes a combination of ingredients harmless to the human body, and has excellent reversibility, so that the photochromic powder reacts with ultraviolet light to thus change in color and the color thereof returns to the original color when the ultraviolet light is not radiated thereon. Accordingly, various cosmetic products capable of being used for the skin, lips, hair, and nails are manufactured using the photochromic powder. Further, various formulations of cosmetic products are manufactured using the photochromic powder so that the color thereof is changed when the cosmetic products are exposed to ultraviolet light, thereby providing various merits, that is, satisfying user demand both for fun and for aesthetic improvement and helping a user create a unique style.

While the above-described embodiments are the most preferred examples of the present invention, the present invention is not limited to the embodiments, and may be applied to hair care products such as hair wax, hair-coating agents, and hair treatments, or to all cosmetic-based products including color cosmetics such as eye shadows and blushers, which are not exemplified above, and it will be apparent to those skilled in the art that various modifications are possible without departing from the technical spirit of the invention.

### INDUSTRIAL APPLICABILITY

The present invention relates to a cosmetic product using photochromic powder and a method of manufacturing the same. More particularly, the present invention relates to a cosmetic product using photochromic powder and a method of manufacturing the same, in which various formulations of cosmetic products used for the skin, lips, nails, and hair are manufactured using a photochromic powder pigment manufactured using ingredients that are harmless to the human body so that the color thereof is changed when the cosmetic products are exposed to ultraviolet light, thereby making it possible to satisfy user demand both for fun and for aesthetic improvement and to help a user create a unique style.

1. A cosmetic product using a photochromic powder, the cosmetic product comprising:
80 to 97 wt % of a base raw material of the cosmetic product; and
3 to 20 wt % of the photochromic powder.

2. The cosmetic product using the photochromic powder of claim 1, wherein the photochromic powder is a micro-encapsulated photochromic powder in which a raw material of the photochromic powder is surrounded by a polyoxymethylenemelamine-styrene copolymer, the raw material of the photochromic powder includes 47 to 49 parts by weight of xylyl phenylethane, 13 to 23 parts by weight of pentaerythrityl tetra-di-t-butyl hydroxyhydrocinnamate, and 2 to 4 parts by weight of a reversible photochromic powder, and a weight ratio of styrene to polyoxymethylenemelamine in the polyoxymethylenemelamine-styrene copolymer is in a range of 1:6 to 6:1.

3. The cosmetic product using the photochromic powder of claim 2, wherein the reversible photochromic powder is any one among trimethyl trifluoromethyl indolinopiperidinyl spironaphtoxazine, indolinotrimethyl spiroindolinenaphtoxazine, trimethyl indolinopiperidinyl spironaphtoxazine, and diphenyl naphthopyran.

4. The cosmetic product using the photochromic powder of claim 1, wherein the base raw material of the cosmetic product is any one base raw material among lip balm, lipstick, lip gloss, tint, hairspray, and nail polish.

5. The cosmetic product using the photochromic powder of claim 4, wherein when the base raw material of the cosmetic product is a base raw material of the lip balm, the lip balm includes 80 wt % of the base raw material of the lip balm and 20 wt % of the photochromic powder.

6. The cosmetic product using the photochromic powder of claim 4, wherein when the base raw material of the cosmetic product is a base raw material of the lipstick, the lipstick includes 85 wt % of the base raw material of the lipstick and 15 wt % of the photochromic powder.

7. The cosmetic product using the photochromic powder of claim 4, wherein when the base raw material of the cosmetic product is a base raw material of the lip gloss or the tint, the lip gloss or the tint includes a combination of 93 wt % of the base raw material of the lip gloss or the tint and 7 wt % of the photochromic powder.

8. The cosmetic product using the photochromic powder of claim 4, wherein when the base raw material of the cosmetic product is a base raw material of the hairspray or the nail polish, the hairspray or the nail polish includes a combination of 97 wt % of the base raw material of the hairspray or the nail polish and 3 wt % of the photochromic powder.

9. A method of manufacturing a cosmetic product using a photochromic powder, the method comprising:
a base-raw-material addition step of adding a base raw material of the cosmetic product, which is synthesized by combining raw materials used to manufacture the cosmetic product, to a container;
a photochromic-powder addition step of adding the photochromic powder manufactured according to claim 1 to a container;
a raw-material-mixing step of mixing the base raw material of the cosmetic product and the photochromic powder that are added using a homomixer; and
a finishing-work step of quantitatively adding the mixed raw materials to a container for cosmetic products, thereby completing manufacture thereof.

10. The method of claim 9, wherein the base raw material of the cosmetic product added in the base-raw-material addition step is any one base raw material among lip balm, lipstick, lip gloss, tint, hairspray, and nail polish.

11. The method of claim 10, further comprising:
when the base raw material of the cosmetic product added in the base-raw-material addition step is the base raw material of the lip balm or the lipstick,
a cooling step of adding raw materials mixed after a raw-material-mixing step to a quick cooler to cool the raw materials.

12. The method of claim 9, wherein the photochromic powder added in the photochromic-powder addition step is manufactured using a process including:
(a) a material addition step of adding materials including xylyl phenylethane, pentaerythrityl tetra-di-t-butyl hydroxyhydrocinnamate, and a reversible photochromic powder, followed by mixing;
(b) a material-mixing step of adding acetone to the materials added in the material addition step and performing mixing so that a weight ratio of the materials and the acetone added is in a range of 2:1 to 1:2;
(c) a microcapsule-material addition step of adding a microcapsule material surrounding an external part of a raw material of the photochromic powder mixed in the material-mixing step;
(d) a curing-agent-addition step of adding a curing agent for curing the microcapsule material;
(e) an agitation step of performing agitation at a high temperature of 100 to 130° C. in order to age microcapsules to which the curing agent is added and an aging step of performing aging in order to stabilize physical properties;
(f) a sterilization and disinfection step of cleaning the microcapsules, which are aged after the aging step, using ethanol;
(g) a photochromic-powder-manufacturing step of drying the cleaned microcapsules and then pulverizing the dried microcapsules into fine particles using an agitator; and
(h) a final inspection step of finally inspecting a cleanliness and a glossiness of the manufactured photochromic powder.

13. The method of claim 12, wherein the reversible photochromic powder is any one among trimethyl trifluoromethyl indolinopiperidinyl spironaphtoxazine, indolinotrimethyl spiroindolinenaphtoxazine, trimethyl indolinopiperidinyl spironaphtoxazine, and diphenyl naphthopyran.

14. The method of claim 13, wherein a microcapsule material added in step (c) is a polyoxymethylenemelamine-styrene copolymer, and a weight ratio of styrene to polyoxymethylenemelamine in the polyoxymethylenemelamine-styrene copolymer is in a range of 1:6 to 6:1.

15. The method of claim 14, wherein a photochromic powder manufactured in step (g) is micro-encapsulated fine particles having a size of 1 to 10 micrometers.

16. The method of claim 15, wherein, in step (a), 47 to 49 parts by weight of xylyl phenylethane, 13 to 23 parts by weight of pentaerythrityl tetra-di-t-butyl hydroxyhydrocinnamate, and 2 to 4 parts by weight of the reversible photochromic powder are added.

17. The method of claim 16, wherein a curing agent added in step (d) is stearic acid.
